(19) United States
(12) Patent Application Publication
(10) Pub. No.: US 2008/0219937 A1
(43) Pub. Date: Sep. 11, 2008

(54) Use of at Least One 2-Alkyl Furan, as Depigmenting or Lightening Active Principle

(75) Inventors: Philippe Msika, Versailles (FR); Nathalie Piccardi, Arceau (FR)

Correspondence Address: FAEGRE & BENSON LLP;PATENT DOCKETING, 2200 WELLS FARGO CENTER, 90 SOUTH SEVENTH STREET, MINNEAPOLIS, MN, 55402-3901, US

(73) Assignee: LABORATOIRES EXPANSCIENCE, COURBEVOIE (FR)

(21) Appl. No.: 12/064,878

(22) PCT Filed: Aug. 30, 2006
(86) PCT No.: PCT/EP2006/065836

§ 371 Date: Feb. 26, 2008

(30) Foreign Application Priority Data
Aug. 30, 2005 (FR) 05/08866

(51) Int. Cl. A61K 8/49; A61Q 17/04; A61Q 19/02

(52) U.S. Cl. 424/59; 424/62

(57) ABSTRACT

The invention concerns the cosmetic use of at least one 2-alkyl furan, as depigmenting active principle, in a depigmenting composition, as well as the associated cosmetic treatment method. The invention also concerns the use of at least one 2-alkyl furan for preparing an active drug as depigmenting agent. The invention further concerns a depigmenting cosmetic composition comprising as depigmenting active principle at least one 2-alkyl furan.

The present invention relates to the use of a cosmetic composition with depigmenting or lightening action, comprising as active principle at least one 2-alkyl furan.

2-alkyl furans, which notably can be extracted from avocado, are furans that are monosubstituted at position 2. Their use has already been described for treating diabetes (international application PCT/FR/2005/001310), cellulitis (international application PCT/FR2005/01311) and obesity (international application PCT/FR2005/01312).

Skin color is due to several substances: hemoglobin from the vessels, carotenoids from the dermis and, especially, melanin from the epidermis. Said melanin is produced by basal layer melanocytes under the action of tyrosinase, copper and oxygen.

Skin melanin is formed by a complex association of eumelanin and pheomelanin.

The biosyntheses of eumelanin and pheomelanin are identical up to dopaquinone (double oxidation of tyrosine by tyrosinase, a copper-containing enzyme). Their biosynthetic pathway then diverges.

Brown eumelanin is an indole-5-6-quinone polymer, whereas pheomelanin, which is responsible for a pink to red hue, is a compound with nearly 10% sulfur and with a cysteinyl dopa polymer structure.

Enzymes other than tyrosinase take part in the genesis and control of melanins: dopachrome oxydoreductase, α-glutamyl transpeptidase, the glutathione system (reductase-peroxidase), dopachrome tautomerase.

Under the effect of exogenous or endogenous stimulation, changes in skin hue can appear. These changes are called dyschromias (hyperchromia and hypochromia).

These changes, which can occur in the epidermis or the dermis, can be due to a variation in the quantity of melanin or in the number of melanocytes.

Hyperchromias are accumulations of melanic pigments, carotenoids or exogenous pigments. Included among hyperchromias are melasma, which is defined as acquired facial hypermelanosis and which can be observed in both sexes and in all races. Melasma more frequently appears among women who use an oral contraceptive or who are pregnant (mask of pregnancy, chloasma).

The mask of pregnancy, or chloasma, appears among women who have high female hormone levels and whose skin is exposed to the sun. Thus, it primarily affects women who are pregnant or who are taking an oral contraceptive. It takes the form of brown pigmented patches, often symmetrical, which are more or less regular in shape.

Cutaneous ageing is also characterized by the appearance of pigment spots. Examples include solar lentigo in the most frequently photo-exposed areas (face, hands, neck and shoulders) and senile lentigo, which are rather large pigment spots that appear on the hands, face and arms of the elderly.

Depigmenting or whitening agents are chemical compounds capable of acting at the tissue, cellular or subcellular level. They act on the formation, transport and color of melanin itself or on the presence of melanocytes (melanocytotoxicity).

In addition, it is necessary to identify and to eliminate the factor that induces hyperpigmentation (ultraviolet radiation, fragrance, combination oral contraceptive) before treating it and to recommend a maximum protection solar protection during the treatment process.

Further, it is possible to eliminate the superficial layers of corneocytes that contain melanin, thus producing physical depigmentation the surface, a treatment that also supports penetration of depigmenting agents.

The motivations for depigmenting the skin are varied. Dark-skinned Africans lighten the skin with traditional or chemical solutions that have significant harmful side effects on the appearance and structure of the skin. Asians lighten facial skin with molecules that act with the least toxicity as possible (arbutin, kojic acid, ascorbic acid).

The treatment of hyperpigmentation spots in white subjects relies on various molecules, the primary of which, hydroquinone, is being increasingly monitored and whose maximum concentration in cosmetics is 2%.

Thus there is a need for compositions with a depigmenting or lightening activity that are well tolerated by the skin.

For this reason, the present invention relates to the use of at least one 2-alkyl furan, as a cosmetic depigmenting or lightening active principle, in a cosmetic composition.

The invention relates to the use of at least one 2-alkyl furan for manufacturing a depigmenting cosmetic composition. The depigmenting cosmetic composition is advantageously intended to reduce and/or eliminate and/or prevent pigment spots or to lighten naturally pigmented skin.

Within the framework of the present invention, the expression “2-alkyl furan” means alkyl furans, monosubstituted at position 2, of general formula (I)

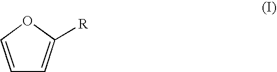

wherein R represents a C₁-C₃₅ alkyl radical, advantageously C₁₀-C₂₂, more advantageously C₁₂-C₂₀, still more advantageously C₁₃-C₁₇; a C₁-C₃₅ alkenyl radical, advantageously C₁₀-C₂₂, more advantageously C₁₂-C₂₀, still more advantageously C₁₃-C₁₇; or a C₁-C₃₅ alkynyl radical, advantageously C₁₀-C₂₂, more advantageously C₁₂-C₂₀, still more advantageously C₁₃-C₁₇, said alkyl, alkenyl and alkynyl radicals possibly being substituted by one or more halogens and/or one or more functions chosen among the functional groups comprising epoxide, hydroxyl (—OH), thiol (—SH), ether (—OR₁), primary amino (—NH₂), secondary amino (—NHR₁), tertiary amino (—NR₁R₂), aldehyde (—CHO), ketone (—COR¹), acetyl (—O—CO—R₁), with R₁ and R₂ representing, independently of each other, a hydrogen atom, a C₁-C₃₅ alkyl radical, advantageously C₁₀-C₂₂, more advantageously C₁₂-C₂₀, still more advantageously C₁₃-C₁₇; or a C₁-C₃₅ alkenyl radical, advantageously C₁₀-C₂₂, more advantageously C₁₂-C₂₀, still more advantageously C₁₃-C₁₇.

Within the framework of the present invention, the term “alkyl” means an unsaturated straight or branched hydrocarbon residue. The term “alkenyl” means a straight or branched hydrocarbon residue comprising unsaturated olefins (double bonds). The term “alkynyl” means a straight or branched hydrocarbon residue comprising at least one triple bond and, optionally, unsaturated olefins.

According to one advantageous embodiment of the invention, said alkyl furans are natural 2-alkyl furans, notably those present in furanic unsaponifiable of avocado, of general formula (I), wherein R represents a radical chosen among the group comprised of the following radicals (*—R):

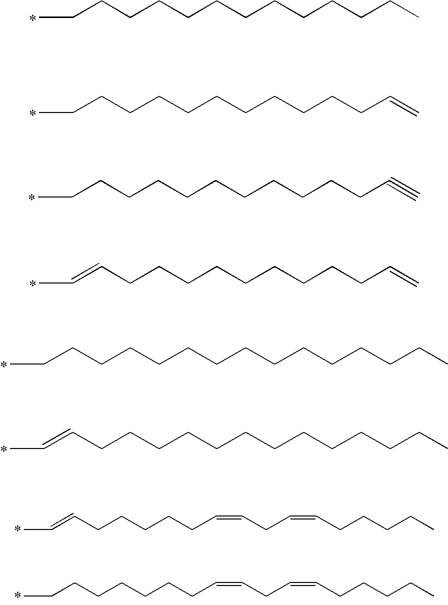

It should be noted that the composition of furanic unsaponifiable of avocado is appreciably different from that of “conventional” unsaponifiable of avocado, known as sterolic unsaponifiable of avocado or sterolic avocado oil. Indeed, conventional unsaponifiable of avocado can contain alkanes, long chain alcohols, triterpene alcohols and sterols (WO99/43298). They are primarily sterolic.

TABLE 1
Comparison of sterolic and furanic
unsaponifiable of avocado

| Unsaponifiable of | Sterolic avocado | Furanic avocado |
|---|---|---|
| avocado | oil | oil |
| Source | Fresh fruit by | Heat dried fruit |
| | centrifugation | by pressure |
| Sterolic fraction | 40-60 | 3-8 |
| H fraction | 0 | 50-80 |
| (furans) | | |
| Other (fatty | 60-40 | ≦15 |
| alcohols, | | |
| tocopherols, | | |
| squalene, etc.) | | |

Thus, international application WO99/43298, which divulges that conventional avocado unsaponifiable can be used in the treatment of stretch marks, is mute as to the therapeutic or cosmetic use of furanic unsaponifiable of avocado.

In furanic unsaponifiable of avocado, 2-alkyl furans account for 30% to 70% by weight compared to the total weight of unsaponifiable.

Avocado oil can contain 2% to 4% by weight of 2-alkyl furans compared to the total weight of oil.

According to one advantageous embodiment of the invention, a purified furanic unsaponifiable fraction of avocado, one that contains 70% to 100% by weight, advantageously 90% to 100% by weight, of 2-alkyl furans compared to the total weight of the fraction, is used to prepare the depigmenting cosmetic composition.

The unsaponifiable is the fraction of a fatty substance that, after the extended action of a basic alkaline, remains insoluble in water and can be extracted by an organic solvent. Five major groups of substances are present in the majority of plant oil unsaponifiables: saturated or unsaturated hydrocarbons, aliphatic or terpene alcohols, sterols, tocopherols, and carotenoid and xanthophyll pigments.

Furanic derivatives of avocado oil are compounds known to those persons skilled in the art. For example, they have been described in Farines, M. et al., 1995, J. of Am. Oil Chem. Soc. 72,473.

Furanic lipid-rich unsaponifiable of avocado has already been described for its use in the manufacture of a medicament having a beneficial and curative action on conjunctive tissue, in particular in the treatment of inflammatory pathologies such as osteoarthritis, periodontitis and scleroderma.

Within the framework of the present invention, the expressions “alkyl furan” and “furanic lipid” are synonymous with furanic unsaponifiable of avocado.

The avocado is advantageously selected among the Hass, Fuerte, Ettinger, Bacon, Nabal, Anaheim, Lula, Reed, Zutano, Queen, Criola Selva, Mexicana Canta, Region Dschang, Hall, Booth, Peterson and Collinson Red varieties, more advantageously the Hass, Fuerte and Reed varieties. Preferably the Hass, Fuerte, Ettinger and Bacon varieties are selected, more advantageously the Hass and Fuerte varieties are selected.

In the purified furanic fraction of unsaponifiable of avocado, as developed by Expanscience Laboratories (see international application WO 01/21605), the 2-alkyl furans represent from 70% to 100% by weight, compared to the weight of the fraction, and the relative weight proportion of each furan identified are given in table 2 below:

TABLE 2

| 2-alkyl furan composition of the purified | |
|---|---|
| furanic fraction of unsaponifiable of | % by |
| avocado | weight |
| 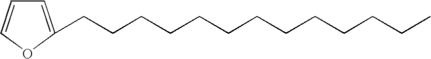 | 3-12 |
| 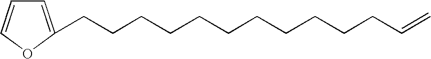 | 1-8 |
| 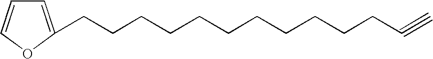 | 1-5 |
| 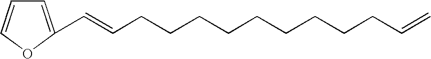 | 1-6 |
| 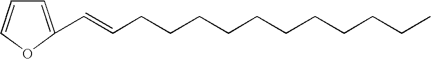 | 5-20 |
| 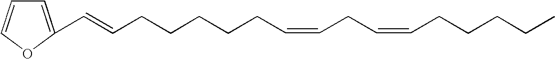 | 10-30 |
| 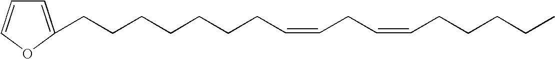 | 35-75 |

In international application WO 01/21605, Expanscience Laboratories implemented a specific method for obtaining a selective extraction of furan lipids of avocado with a content of more than 80%, even approaching 100%, by weight of furanic lipids.

Said method comprises the steps of preparation of unsaponifiable of avocado and then subjecting the unsaponifiable of avocado to a molecular distillation step by using the means of regulated temperature and pressure to obtain a distillate comprising primarily furanic lipids of avocado.

Preferably, unsaponifiable of avocado is prepared from fruit pre-treated thermally before the extraction of oil and saponification, as described in particular in patent application FR 2,678,632. Said thermal treatment consists of the controlled drying of the fruit, preferably fresh, for at least four hours, advantageously between 24 and 48 hours, at a temperature preferably of at least roughly 80° C., more preferably between roughly 80° C. and roughly 120° C., with temperature and drying time being interdependent.

Before its saponification, the oil can be pre-enriched in unsaponifiable by separating a majority of the components of unsaponifiable recovered in a concentrate. Various methods can be used, such as cold crystallization, fluid-fluid extraction or molecular distillation. Molecular distillation, which is particularly preferred, is carried out advantageously at a temperature between roughly 180° C. and roughly 230° C. while maintaining pressure between 10⁻³ mmHg and 10⁻² mmHg.

Unsaponifiable of avocado, obtained as described above, is then subjected to a molecular distillation step. Said molecular distillation step is carried out at a temperature that can be regulated between 100° C. and 160° C. and at a pressure that can be regulated between 10⁻³ mmHg and 5×10² mmHg. In particular, temperature is regulated between 100° C. and 140° C. and pressure is regulated between 10⁻³ mmHg and 5×10⁻² mmHg to obtain a distillate primarily comprising furanic lipids of avocado.

In international application WO 04/016106, Expanscience Laboratories developed a method for obtaining, with increased yield, unsaponifiable of avocado rich in furanic lipids, namely at concentrations of 50% to 80%, with low concentrations of heavy products and peroxides.

Said method comprises the following successive steps:
(1) a step of controlled dehydration of fresh avocados or those having undergone preliminary transformations, carried out at a temperature between −50° C. and +75° C.,
(2) a step of extracting the oil from the dried fruits,
(3) a step, alternatively,
   a. of thermal treatment of the extracted oil at a temperature that can vary from 80° C. to 150° C., optionally in an inert atmosphere, and then of a step of concentrating the oil in its unsaponifiable fraction, or
   b. of concentrating the oil in its unsaponifiable fraction, and then a thermal treatment at a temperature that can vary from 80° C. to 150° C., optionally in an inert atmosphere, followed by
(4) a step of saponification and extraction of unsaponifiable.

Dehydration, as carried out in step (1) of the method, means, more generally, all techniques known to those persons skilled in the art for extracting water from a compound. Preferred techniques include drying in ventilated driers, in thin layers and under a flow of hot air, at a temperature between 70° C. and 75° C., for 8 to 36 hours.

The thermal treatment step used in step (3)a or (3)b can take place in the presence or absence of an acid catalyst, preferably an alumina acid catalyst. The concentration step of step (3)a or (3)b can be cold crystallization or molecular distillation.

Alternatively, the preparation of unsaponifiable fraction of avocado comprised of alkyl furans can involve raw materials that are by-products of avocado oil extraction methods, namely oils obtained from fresh fruits that are not dried beforehand.

Such by-products include, without being limiting in any way, i) fatty phases and ii) aqueous phases arising from centrifugation, or those arising from enzymatic methods such as, in particular, enzymatic pre-digestion of plant cells in the pulp, in order to facilitate the release of lipids from the fruit. Solid residues from centrifugation (pellets) extracted from crude oil washes can also constitute a desirable raw material.

In addition, another alternative involves the use of frozen pulp from previously peeled and stoned fruits. Similarly, avocado oil deodorization exhaust also constitutes a source of unsaponifiable of avocado and alkyl furans.

In addition, avocado oil meal co-produced during cold mechanical pressurization of the fruits (fresh or dried) or during fluid-solid extraction of avocado oil using a solvent can also constitute a ready to use raw material alternative.

Finally, although low in oil content, avocado pits can constitute a potential source of avocado lipids, most notably alkyl furans.

Within the framework of the present invention, the depigmenting cosmetic composition advantageously comprises 0.001% to 25% of 2-alkyl furans by weight, more advantageously 0.01% to 10% of 2-alkyl furans by weight, even more advantageously 0.1% to 5% of 2-alkyl furans by weight, compared to the total weight of the cosmetic composition and a cosmetically acceptable medium.

The compositions according to the invention can contain other cosmetic actives with depigmenting action, providing a complementary or synergistic effect. The 2-alkyl furans can be associated with depigmenting agents, known to those persons skilled in the art, such as hydroquinone and its derivatives, arbutin, retinoic acid, retinol, retinaldehyde, kojic acid, azelaic acid, vitamin B3 or PP, resorcinol derivatives, resveratrol, extract of licorice or of white mulberry, alpha-lipoic acid, linoleic acid, cation chelators such as EDTA (ethylenediaminetetraacetic acid), and soya extracts.

The 2-alkyl furans can also be combined with cosmetic antioxidant agents, providing a complementary or synergistic effect. Examples of antioxidant agents most notably include vitamin C, vitamin E, polyphenols (in particular those extracted from green tea or grapes or pine) and sulfur derivatives.

The 2-alkyl furans can also be combined with cosmetic depigmenting agents such as Sepiwhite® (N-undecylenoyl-L-phenylalanine, sold by Seppic), providing a complementary or synergistic effect.

According to another embodiment of the invention, the cosmetic compositions according to the invention also contain, possibly with a synergistic effect, at least one UVA and UVB sun filter or screen, such as the mineral and/or organic screens or filters known to those skilled in the art, who will establish the best choice and concentration according to the required degree of protection.

The according to the invention cosmetic compositions can also contain exfoliating agents such as alpha hydroxy acid, salicylic acid and derivatives of same in ester form, for example.

Lastly, cosmetic compositions the according to the invention can also contain anti-inflammatory or soothing agents, cutaneous desensitizing agents such as NSAIDs (non-steroidal anti-inflammatory drugs), dermocorticoids, PPAR (peroxisome proliferator-activated receptor) agonists, liquorice derivatives, bisabolol, glycosylated or non-glycosylated isoflavones (of soya for example), palmitoylethanolamide, unsaponifiables containing phytosterols and vitamins E, COX (cyclooxygenase) and/or LOX (lipoxidase) inhibitors, thermal spring water, sea water or water reconstituted from exogenous trace elements.

The cosmetic composition according to the invention comprises a cosmetically acceptable medium, i.e., a medium that is compatible with the skin. Advantageously it can be provided in any galenical form typically used for topical administration, in particular in the form of an aqueous solution, hydroalcoholic or oily; an oil-in-water or water-in-oil or multiple emulsion; an aqueous or oily gel; a solid, liquid or paste anhydride; a dispersion of oil in an aqueous phase using spheroids (nanospheres, nanocapsules, lipid vesicles); a trans-dermal device; or in any other form for topical application.

Said composition can be more or less fluid and can take the form of a white or tinted cream, an ointment, a milk, a lotion, a serum, a paste, a foam or a gel. Optionally, it can be applied to the skin in the form of an aerosol. It can also have a solid form, for example in the form of a stick. It can also be applied by means of a patch.

The composition according to the invention can also contain additives typically used in the fields of cosmetics, dermatology and/or pharmacology, such as stabilizers, preservatives, antioxidants, solvents, fragrances, chelating agents, odor absorbers, chemical or mineral filters, mineral pigments, surfactants, polymers, silicone oils and dyes.

The invention also relates to a method of cosmetic treatment for reducing and/or eliminating pigment spots, characterised in that a cosmetic composition comprising at least one 2-alkyl furan, as a cosmetic depigmenting active principle, is applied by topical route. This cosmetic treatment method makes the color of the skin more uniform. The cosmetic composition is advantageously that described above.

Pigment spots can include, but are in no way limited to, age spots, UV induced spots or phototoxicity spots (fragrances, drugs, exogenous toxins, burns) or chloasmas.

The invention also relates to a method of cosmetic treatment for lightening the skin, characterised in that a cosmetic composition comprising at least one 2-alkyl furan, as a cosmetic depigmenting active principle, is applied by topical route. The cosmetic composition is advantageously that described above.

The depigmenting properties of 2-alkyl furans can, according to another embodiment of the invention, lead to the use of at least one 2-alkyl furan as an active principle for preparation of an active medicament as a depigmenting agent.

The 2-alkyl furan used to manufacture the medicament is advantageously that described above. It can be used in combination, possibly with a synergistic effect, with at least one other depigmenting agent as defined above and/or at least one organic or mineral sun filter and/or an anti-inflammatory agent.

The optimal modes of administration, dosing and galenical forms of the compounds and compositions according to the invention can be determined according to the criteria generally considered in establishing a cosmetic and/or dermatological and/or pharmaceutical treatment, adapted to a patient according to skin type, for example.

The following examples illustrate the present invention.

### EXAMPLE 1

### Depigmenting Skin Cream No. 1

| INGREDIENTS | % w/w |
|---|---|
| Water | QSP 100 |
| Di-C₁₂₋₁₃ alkyl malate | 10.000 |
| Hydrogenated polydecene | 5.000 |
| Oryza Sativa starch | 4.000 |
| Cetearyl alcohol | 3.200 |
| Glycerin | 3.000 |
| Hydrogenated coco-glycerides | 3.000 |
| Sorbitan stearate | 3.000 |
| Undecylenoyl phenylalanine | 2.000 |
| Ascorbyl glucoside | 2.000 |
| Tromethamine | 1.340 |
| Ceresin | 1.000 |
| Cetearyl glucoside | 0.800 |
| Fragrance | 0.500 |
| Xanthan gum | 0.400 |
| Potassium cetyl phosphate | 0.400 |
| Sclerotium gum | 0.300 |
| Sodium hydroxymethylglycinate | 0.200 |
| 2-Alkyl furan | 0.100 |
| Tocopherol | 0.100 |

### EXAMPLE 2

### Depigmenting Skin Cream No. 2

| INGREDIENTS | % w/w |
|---|---|
| Water | QSP 100 |
| Di-C₁₂₋₁₃ alkyl malate | 10.000 |
| Hydrogenated polydecene | 5.000 |
| Oryza Sativa starch | 4.000 |
| Cetearyl alcohol | 3.200 |
| Glycerin | 3.000 |
| Hydrogenated coco-glycerides | 3.000 |
| Sorbitan stearate | 3.000 |
| Tromethamine | 1.340 |
| Ceresin | 1.000 |
| Cetearyl glucoside | 0.800 |
| Fragrance | 0.500 |
| Xanthan gum | 0.400 |
| Potassium cetyl phosphate | 0.400 |
| Sclerotium gum | 0.300 |
| Sodium hydroxymethylglycinate | 0.200 |
| 2-Alkyl furan | 0.200 |

### EXAMPLE 3

### Depigmenting Spray, Sunscreen Protection Factor (SPF) 30

| Ingredients | % w/w |
|---|---|
| Water | QSP 100 |
| Pentaerythrityl tetraoctanoate | 15 to 30 |
| Titanium dioxide | 1 to 10 |
| Cyclomethicone | 1 to 10 |
| Zinc oxide | 1 to 10 |
| Methylene bis-benzotriazolyl | 1 to 5 |
| tetramethylbutylphenol | |
| (C₁₂-C₁₅)alkyl benzoate | 1 to 10 |
| 4,5,7-Trihydroxyisoflavone | 0.01 to 10 |
| Glycerin | 1 to 10 |
| Dicaprylyl ether | 1 to 10 |
| Cyclopentasiloxane | 1 to 10 |
| Ethylhexyl dimethicone ethoxy glucoside | 1 to 10 |
| Propylene glycol dioctanoate | 1 to 10 |
| Sodium chloride | 1 to 5 |
| PEG-45/dodecyl glycol copolymer | 1 to 5 |
| PEG-30 dipolyhydroxystearate | 1 to 5 |
| Unsaponifiable soya oil | 1 to 5 |
| Dextrin palmitate | 1 to 5 |
| 2-Alkyl furan | 0.01 to 3 |
| Extract of Aloe barbadensis | 0.2 |
| Preservatives | QS |
| Zinc gluconate | 0.08 |

### EXAMPLE 4

### Depigmenting Skin Cream, SPF 50

| Ingredients | % w/w |
|---|---|
| Water | QSP 100 |
| Pentaerythrityl tetraoctanoate | 15 to 30 |
| Titanium dioxide | 1 to 10 |
| Cyclomethicone | 1 to 10 |
| Zinc oxide | 1 to 10 |
| (C₁₂-C₁₅)alkyl benzoate | 1 to 10 |
| Undecylenoyl phenylalanine | 0.5 to 2 |
| 2-Alkyl furan | 0.01 to 10 |
| 4,5,7-Trihydroxyisoflavone | 0.01 to 10 |
| Glycerin | 1 to 10 |
| Dicaprylyl ether | 1 to 10 |
| Cyclopentasiloxane | 1 to 10 |
| Ethylhexyl dimethicone ethoxy glucoside | 1 to 10 |
| Propylene glycol dioctanoate | 1 to 10 |
| Sodium chloride | 1 to 5 |
| PEG-45/dodecyl glycol copolymer | 1 to 5 |
| PEG-30 dipolyhydroxystearate | 1 to 5 |
| Unsaponifiable soya oil | 1 to 5 |
| Dextrin palmitate | 1 to 5 |
| Preservatives | QS |
| Extract of Aloe barbadensis | 0.2 |
| Zinc gluconate | 0.08 |

### EXAMPLE 5

### Depigmenting Skin Cream No. 3

| INGREDIENTS | % w/w |
|---|---|
| Water | QSP 100 |
| Di-C₁₂₋₁₃ alkyl malate | 10.000 |
| Hydrogenated polydecene | 5.000 |
| Oryza Sativa starch | 4.000 |
| Cetearyl alcohol | 3.200 |
| Glycerin | 3.000 |
| Hydrogenated coco-glycerides | 3.000 |
| Sorbitan stearate | 3.000 |
| Ascorbyl glucoside | 2.000 |
| Tromethamine | 1.340 |
| Ceresin | 1.000 |
| Cetearyl glucoside | 0.800 |
| Fragrance | 0.500 |
| Xanthan gum | 0.400 |
| Potassium cetyl phosphate | 0.400 |
| Bisabolol | 0.300 |
| Sclerotium gum | 0.300 |
| Sodium hydroxymethylglycinate | 0.200 |
| 2-Alkyl furan | 0.100 |
| Tocopherol | 0.100 |

1-14. (canceled)

15. A method for depigmenting the skin of a person in need thereof comprising the administration to said person of at least one 2-alkyl furan in a composition.

16. The method of claim 1, for reducing pigment spots.

17. The method of claim 1, for eliminating pigment spots.

18. The method of claim 1, for preventing pigment spots.

19. The method of claim 15, wherein the 2-alkyl furan is of general formula (I) 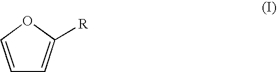
wherein R represents a C₁-C₃₅ alkyl radical, a C₁-C₃₅ 20 alkenyl radical or a C₁-C₃₅ alkynyl radical.

20. The method of claim 19, wherein said alkyl, alkenyl and alkynyl radicals are substituted by one or more halogen atoms.

21. The method of claim 19, wherein said alkyl, alkenylr and alkynyl radicals are substituted by one or more functional groups comprising epoxide, hydroxyl (—OH), thiol (—SH), ether (-0R₁), primary amino (—NH₂), secondary amino (—NHR₁), tertiary amino (—NR₁R₂), aldehyde (—CHO), ketone (—COR₁) or acetyl (-0-CO—R₁), with R₁ and R₂ representing, independently of each other, a hydrogen atom, a C₁-C₃₅ alkyl radical or a C₁-C₃₅ alkenyl radical.

22. The method of claim 19, wherein said alkyl, alkenyl and alkynyl radicals are substituted by one or more halogen atoms or functional groups comprising epoxide, hydroxyl (—OH), thiol (—SH), ether (—OR₁), primary amino (—NH₂), secondary amino (—NHR₁), tertiary amino (—NR₁R₂), aldehyde (—CHO), ketone (—COR₁), or acetyl (—O—CO—R₁), with R₁ and R₂ representing, independently of each other, a hydrogen atom, a C₁-C₃₅ alkyl radical or a C₁-C₃₅ alkenyl radical.

23. The method of claim 15, wherein the 2-alkyl furan is a natural 2-alkyl furan of general formula (I), wherein R comprises the following radicals (*—R):

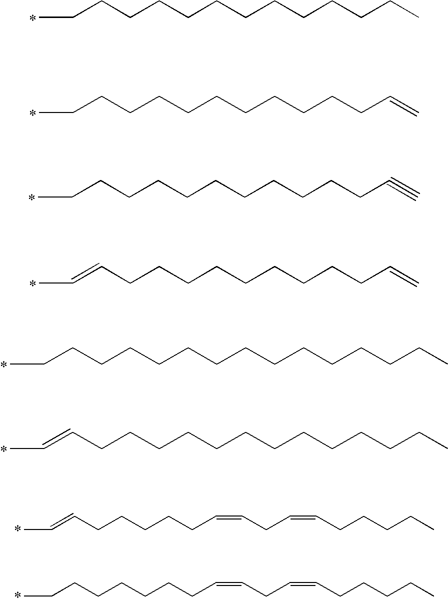

24. The method of claim 23, wherein said natural 2-alkyl furan is present in furanic unsaponifiable of avocado.

25. The method of claim 15, wherein the composition comprises between 0.001% and 25% 2-alkyl furan by weight, compared to the total weight of the composition and a cosmetically acceptable medium.

26. The method of claim 15, wherein the composition further comprises another depigmenting cosmetic active principle.

27. The method of claim 15, wherein the composition 15 further comprises at least one organic or mineral sun filter.

28. The method of claim 15, wherein the composition is applied by a topical route.

29. The method of claim 16, wherein the pigment spots are age spots, sun-induced spots, or spots related to phototoxicity or chloasmas.
